(19) United States
(12) Patent Application Publication
(10) Pub. No.: US 2007/0292489 A1
(43) Pub. Date: Dec. 20, 2007

(54) METHOD FOR TREATMENT OF WOUND TREATMENT USING AGANOCIDES

(76) Inventors: Mansour Bassiri, Novato, CA (US); Ramin Najafi, Novato, CA (US); Behzad Khosrovi, El Cerrito, CA (US)

Correspondence Address: HELLER EHRMAN LLP, 275 MIDDLEFIELD ROAD, MENLO PARK, CA, 94025-3506, US

(21) Appl. No.: 11/763,368

(22) Filed: Jun. 14, 2007

Related U.S. Application Data
(60) Provisional application No. 60/813,805, filed on Jun. 14, 2006.

(51) Int. Cl. A61K 31/66; A61K 31/185; A61L 15/00

(52) U.S. Cl. 424/445; 514/561; 514/553; 514/114

(57) ABSTRACT

Provided herein is a method for the treatment of a wound using a wound treatment regimen for treating a chronic wound, the regimen comprising: a) applying reduced pressure to the wound; and b) treating the wound with a substantially non-toxic, non-irritating organic aganocide composition while applying reduced pressure to the wound.

### CROSS-REFERENCE TO RELATED APPLICATION

This is a non-provisional application 37 C.F.R. §1.53(b), claiming priority under 37 C.F.R. §119(e) to U.S. Provisional Patent Application Ser. No. 60/813,805, filed on Jun. 14, 2006, the entire disclosure of which is hereby expressly incorporated by reference.

The present invention relates to a method for treating a wound by applying reduced pressure to the wound, while treating the wound with a substantially non-toxic, non-irritating organic aganocide composition before, during or after applying reduced pressure to the wound.

### BACKGROUND OF THE INVENTION

The effective treatment of open wounds that are too large to spontaneously close has long been a significant problem to medical practitioners. Some details explaining these difficulties can be found in U.S. Pat. Nos. 5,636,643 and 5,645,081, the entire disclosures of which are incorporated herein by reference. Some of the factors preventing wound closure of permanent open wounds are bacterial infection of the wound, insufficient blood circulation and the presence of necrotized tissue. Louis C. Argenta and Michael J. Morykwas introduced the vacuum-assisted wound closure (VAC) system in 1997 for the treatment of pressure ulcers and other chronic wounds (Ann Plast Surg 1997; 38:553-562 and 563-577). This system is based on the application of negative pressure to the wound, resulting in arteriolar dilatation and promoting granulation tissue proliferation and resulting in substantially improved wound healing conditions.

As concerns bacterial infection of an open wound, a bacterial count of 10⁵ or more per gram of tissue is defined as infected. It is known that wound tissue that is infected will not be susceptible to wound closure. Therefore, a number of approaches have been taken to reduce bacterial infection. For example, iodine or silver salts have been used to eliminate bacterial infection that would prevent wound closure. However, many patients are sensitive or allergic to iodine treatment of wounds, and even more so to silver salts. In addition, silver salts have not been very effective, and have reported to cause argyria, a permanent staining of the skin. In order to mitigate these effects, povidone-iodine has been used, but some patient sensitivity or allergic reactions will remain. Dakin's solution (a buffered solution of NaOCl of a mild basic pH) is also used frequently for the treatment of open wounds but is not very effective. In addition, Dakin's solution is cytotoxic, which impedes wound healing. Therefore, wound healing experts discourage the use of Dakin's solution. It has been also proposed to apply antibiotics topically, but the resistance to treatment with antibiotics are a standing and lingering concern.

More recently, organic aganocides have been proposed such as N-halo or N,N-dihalotaurine derivatives to substantially reduce bacterial count in the treatment of open wounds (U.S. Pat. No. 6,426,066 and U.S. Publication No. 2004/0137078 A1).

Tatjana M. Fleck et al. have proposed applying negative pressure for the treatment of deep sternal wound infections after cardiac surgery and have described debridement with removal of all necrotic tissue and irrigation with dilute povidone-iodine solution and H₂O₂ before and after application of the negative pressure (T. M. Fleck et al. in Ann Thorac Surg 2002; 74:1596-200). However, as explained before, certain patients are allergic and sensitive to povidone-iodine treatment.

### SUMMARY OF THE INVENTION

It is understood that any aspect or feature of the present invention whether characterized as preferred or not characterized as preferred may be combined with any other aspect or feature of the invention, whether such other feature is characterized as preferred or not characterized as preferred. For example, a feature described as preferred, for example a pH range, or a specific pH for a particular composition may be combined with another not preferred feature, for example, a NaCl concentration of 1.2%, without deviating from the present invention.

### DEFINITIONS

“Aganocide” as used herein, refers to N-halo or N,N-dihalo amino acids, or N-halo- or N,N-dihalo amino acid sources. See “N-Halo or N,N-Dihalo Amino Acid Source” below.

“Atraumatic wiping” refers to removal of undesirable matter from or around the wound or skin around the wound without causing trauma either to newly formed tissue or the skin around the wound.

“Bacterial load” refers to the bacterial count in a wound determined by wound biopsy.

“Biopsy” refers to a method of assessing the infection of a wound or ulcer.

“Cell-compatible composition” is an aganocide composition or a NaCl containing composition with acceptable cytotoxicity and is physiologically acceptable for therapeutic applications.

“Combination treatment regimen” is a treatment regimen applying vacuum-assisted wound treatment with use of an aganocide as described herein.

“Debridement” refers to the removal of tissue in and around the wound that may negatively affect wound healing or closure.

“Exudate” refers to a liquid oozing out from a wound that may affect wound healing in a positive or negative sense.

“Granulation” refers to a secondary intention wound as defined below that is vasularizing and healing from the bottom up. Granular appearance (bumpiness) of the wound is not necessary for a wound to be classified as granular.

“N-Halo or N,N-dihalo amino acid source” refers to a composition that releases an N-halo- or N,N-dihalo amino acid for example, a salt, such as the sodium, pottasium, magnesium or calcium salt, that, when treated with an acid releases the corresponding N-halo- or N,N-Dihalo Amino Acid. As an alternative for example, a N,N-dihalo amino acid source may be a polymeric matrix that is stable toward the N-Halo or N,N-dihalo amino acid (or aganocide) but gradually releases the N-Halo or N,N-dihalo amino acid into the wound.

“Long-term application” is differentiated from “Short-term application” and refers to the extended application of a component of the regimen to the wound or ulcer. As a rule it will be an application for more than 120 minutes, preferably for more than 6 hours.

“Precede” refers to a step, procedure or application in the regimens described herein that goes before a step, procedure or application that follows.

“Regimen” is a systematic topical therapy plan or schedule designed to promote the healing of wounds or ulcers.

“Removal of a composition” refers to the separation of a matter or a composition from the wound, ulcer or skin around the wound, for example, of a component of the regime described.

“Saline” is a NaCl containing composition, mostly in form of a solution that is physiologically acceptable to the mammalian skin in the area of the wound or ulcer; it may also contain other alkali or earth-alkali ions.

“Secondary intention wound” is a wound in which some tissue has been lost and healing is predominantly done by cleaning and allowing self-regeneration.

“Short-term application” is differentiated from “long-term application” and refers to the temporary application of a component of the regimen to the wound or ulcer. As a rule it will be an application for not more than 120 minutes.

“The size of the wound and/or the bacterial load have been reduced sufficiently” refers to the wound healing progress achieved after application of the regimen herein described to such an extent that the wound-healing would continue even if the regimen would be discontinued.

“Time intervals appropriate to patients in need of wound care” refer to the long-term application of a component or composition of the regimen beneficial in terms of promoting wound healing, enhancing patient compliance and in a health care facility coincide with patient visits by physicians, nurse practitioners, nurse assistants and personnel capable of performing the wound care required by the various regimens described herein.

“Time kill” refers to an in vitro measure of how fast a given antimicrobial can kill test bacteria.

“Treatment cycle” refers to the completion of a regimen after a number of repetitions of the alternating steps, as defined herein.

“Wiping” is a specific removal process by rubbing or cleaning or drying, in general without causing infection of a wound or ulcer.

“Wound or ulcer” is a break in external skin accompanied by loss of skin surface tissue, disintegration and necrosis of epithelial tissue.

“Wound closure is occurring” refers to the wound healing progress achieved after application of the regimen herein described. In general, such progress is achieved when the fraction of open wounds is reduced by at least 70% relative to the open wounds existing prior to administration of the regimens described herein.

“Wound dressing” refers to a protective or therapeutic material applied to a wound minimizing infection and producing an environment beneficial to promoting wound healing.

“Wound tracing” refers to various methods or a specific method to measure and/or evaluate wound surface area.

In accordance with the present invention we provide a new method for treating a wound, in particular a chronic wound, by applying reduced pressure to the wound, while treating the wound with a substantially non-toxic, non-irritating member of an organic aganocide composition selected from the group consisting of an N-Halo amino acid, an N,N-dihalo amino acid, a source of N-Halo amino acid, a source of N,N-dihalo amino acid, and mixtures thereof, in conjunction with, or before, during or after applying reduced pressure to the wound.

The method may use any device or combination of devices suitable for delivering the organic aganocide compositions intermittently to the wound and for applying negative pressure (i.e., vacuum) to the wound. Devices include the one described in U.S. Pat. Nos. 5,636,643 or 5,645,081 or a device as described in Ann Thorac Surg 2002; 74:1596-1600, (VAC system provided by KCI Inc., San Antonio, Tex.), or in KCI's V.A.C.®^{instill} Brochure and the Versatile 1™ Wound Vacuum System as described on the website of BlueSky Medical (currently http://www.blueskymedical.com/).

However, the organic aganocide composition may also be delivered to the wound before, or after applying the vacuum device or system, or between two or more applications of the vacuum system to the wound. In this embodiment, the organic aganocide composition may be sprayed or poured on the wound or applied by injection, by irrigation, by dripping it on the wound, or gentle rubbing it onto the wound, or touching the wound, or by any form of contact that achieves its application onto the wound without negatively affecting wound healing. In one particular aspect, it is understood that in certain applications, the vacuum system may not be used for aganocide delivery, but only for application of the vacuum.

The new treatment method is effective to promote healing of difficult healing wounds. The aganocide composition and reduced pressure are applied to a wound for a sufficient time and with sufficient concentration of the aganocide and sufficiently reduced pressure to facilitate wound closure. This can be achieved by many different approaches as described in more detail below.

### DETAILED DESCRIPTION

Most frequently in the treatment regimen described herein, before applying negative pressure, necrotic tissue is removed and the organic aganocide composition applied to the wound. Sometimes, in case of severe wounds, before application of the aganocide composition, the wound may be traced to determine its surface area. Biopsy may also be used to ascertain the degree of infection of the wound. The aganocide composition may be applied by spraying it on the wound, by injection, by pouring it on the wound, by irrigation, by using a soft carrier containing the aganocide and contacting or touching the wound lightly with the carrier containing the aganocide. Thereafter, a cover, such as an impermeable cover is fitted on the wound to cover and enclose the wound. As described in more detail below, a suitable porous material may be placed into or over the wound before the impermeable cover is fitted on the wound. The impermeable cover is configured to maintain reduced pressure at the site of the wound. The cover is connected with a vacuum pump to apply a vacuum or reduce pressure to the wound. The cover is then sealed, for example, with drape to provide an airtight seal. Thereafter suction is applied to collect wound fluid that is removed by suction.

The organic aganocide composition should be applied in concentration of up to 10,000 ppm. For the N-halogenated or N,N-dihalogenated amino acid aganocide or a derivative thereof, for example, as described in WO0222118 W. Gottardi, U.S. Publication No. 2005/0065115 A1 and U.S. Ser. No. 11/339,987 filed Jan. 25, 2006, the concentration to be chosen should be at least 100 ppm, because those aganocide have a mild antibacterial action. The mono-halogenated amino acids (i.e. N-halo amino acids), such as N-chlorotaurine, have the mildest antibacterial action among all N-halogenated amino acids, milder than the antibacterial action of N,N-dichlorotaurine or N,N-dichloro-2,2-dimethyl taurine. Therefore the higher concentration ranges may be used for the N-monohalogenated amino acids. Preferably, the effective amount of an organic aganocide such as an N- halogenated amino acid is from about 100 to about 10,000 ppm. Even more preferred, the effective amount is from about 200 to about 2,000 ppm. Most preferably, the effective amount is from about 400 to about 1,000 ppm.

The pH of the aganocide compositions which are preferably applied in form of aqueous solutions is not critical as long it is acidic to neutral. A broad pH range from about 2 to about 7 may be a useful range. A more preferred pH range is between about 3 and about 7, and a pH range between about 3.5 to about 7 may be most preferred. If an acidic pH range is the best choice for a given wound condition, a pH between about 3.5 to about 7 is preferable. Even more preferred may be a range between about 3.5 and about 6.5.

If the organic aganocide composition is used in the form of a solution, then water or a lower alcohol (such as ethanol or isopropanol) or mixtures thereof may be the best choice for the vehicle. If an aqueous composition is being used it would be most advantageous to use a saline composition (for example, NaCl) in amounts of about 0.4 to about 1.2% by weight of NaCl, or the equivalent of a similar salt such as KCl, because such compositions would be even less irritating.

The method described herein includes at least one step of applying the organic aganocide to the wound and its removal or replacement, for example, before application of the vacuum. In one particular embodiment, the aganocide may also be applied while the vacuum is on by a device having a separate port for delivering the aganocide composition with a shut-off valve which would initially be in a closed position while the wound is under reduced pressure. Then a valve controlling the port to the vacuum pump would be shut off and the valve controlling delivery of the aganocide composition would be slowly and carefully opened to apply a certain often pre-determined amount of aganocide to the wound. After shutting off the aganocide delivery valve, the vacuum can be re-applied, resulting in removal of wound exudate and excess aganocide.

A preferred embodiment comprises at least two applications of the organic aganocide, or one application of aganocide followed by saline treatment and at least one application of reduced pressure, to debride the wound and reduce bacterial count, for example, before the application of the vacuum, and after the vacuum treatment has been discontinued. This method would have the benefit that bacterial count of the wound is reduced before negative pressure (vacuum) is applied that will result in an even further reduced bacterial count. After the vacuum has been shut off, further debridement with the aganocide or with saline may be performed.

An even more preferred treatment involves an intermittent treatment regimen, for example as follows:
1. Deliver the aganocide composition to the wound under sterile conditions
   a) by spraying;
   b) by injection;
   c) by pouring the composition on the wound; and/or
   d) by irrigation;
2. Leave composition on the wound for 1 second to 120 minutes or 5 minutes to 60 minutes;
3. Remove the composition by gentle atraumatic wiping or reduced pressure (vacuum);
4. Apply reduced pressure for more extended period (for example, 1 minute-12 hrs);
5. Shutting off the vacuum;
6. Reapply the composition (aganocide or saline); and optionally
7. Repeat the treatment cycle

A complete treatment cycle comprises at least two applications of the organic aganocide composition and lasts at least 24 hours during which negative pressure is applied.

Alternatively, prior to applying negative pressure the aganocide composition applied to the wound may be removed by changing the dressing, changing the dressing and wiping, delivery of fresh aganocide composition and rinsing off aganocide residues, such as by using water and/or saline solution, from previously applied aganocide or pouring on new aganocide composition and rinsing off aganocide residues from previously applied aganocide.

A typical treatment regimen will last from at least 12 hours and can last up to several weeks and will comprise multiple treatment cycles until the status of sufficient wound closure has been achieved to enable self-healing of the wound. It is preferred to achieve wound closure or the status of self-healing in two weeks or less time.

The reduced pressure to be applied may range from about 0.01 to about 0.99 atmospheres, more preferably from about 0.03 to about 0.8, or from about 0.03 to about 0.4 atmospheres. A preferred reduced pressure applies at least 0.11 or 0.136 atm to the wound. In certain applications, it is beneficial to provide the reduced pressure in an intermittent or cyclic manner which may be achieved by manual or automatic control of the vacuum system. Details of the VAC treatment regimen are described in U.S. Pat. No. 5,636,643 and U.S. Pat. No. 7,216,651.

Preferred aganocides are N-chlorotaurine or N,N-dichlorotaurine and their salts, and N-halo- or N,N-dihaloamino acids of the formula (I)
A-C(R¹R⁰)R(CH₂)ₙ—C(YZ)—X′ (I)

or a derivative thereof. In this formula A is hydrogen, HalNH— or Hal₂N— wherein Hal is halogen selected from the group consisting of chloro and bromo; R is a carbon carbon single bond or a divalent cycloalkylene radical with three to six carbon atoms, R¹ is hydrogen, lower alkyl or the group —COOH; R⁰ is hydrogen or lower alkyl; n is 0 or an integer from 1 to 13, or R¹ and R⁰ together with the carbon atom to which they attach form a (C₃-C₆)cycloalkyl ring; Y is hydrogen, lower alkyl or —NH₂ or —NHal₂; and Z is hydrogen or lower alkyl; and X′ is hydrogen, —COOH, —CONH₂, —SO₃H, —SO₂NH₂ or —P(═O)(OH)₂. The derivatives include salts.

Preferred are the N,N-dichloroamino acids of the above formula I in which R¹ and R⁰ are not hydrogen, and R¹ and R⁰ both are preferably lower alkyl, most preferably methyl. One of the most preferred aganocides is N,N-dichloro-2,2-dimethyl taurine (2-dichloroamino-2-methylpropanesulfonate) or its sodium salt. Further potentially useful aganocides are the compounds described in U.S. Pat. No. 4,386,103 Pogany, the disclosure of which is incorporated herein by reference in its entirety.

In order to apply the VAC system to a wound pretreated with an organic aganocide composition, generally first a porous material dressing (one or more sponges or sterile pieces of foam, for example, an open-cell polyester or polyether, for example a polyurethane ester or ether) with a pore size ranging from about 400 to 600 micrometers to maximize tissue growth is placed on the wound. Embedded in the foam or sponge is an evacuation tube with various ports that communicate with the foam to control the reduced pressure, to ensure equal distribution of the reduced pressure to all parts of the wound and to prevent wound overgrowth. The shape of the sponge or foam may be fashioned to the specific geometry of the wound. The foam or sponge may also be pretreated with an aganocide described herein, or the aganocide or a precursor of the aganocide may be bound chemically to the foam or sponge, for example, to gradually release aganocide into the wound fluid while it is under negative or under atmospheric pressure.

The wound is then covered with an adhesive tape to form an airtight system. The evacuation tube is connected to a collection device, such as a trap for collecting exudate and any matter debrided from the wound. The trap, in turn, is connected to an adjustable vacuum pump, which is able to generate the desired negative pressure. It is preferred to equip the collection trap with a non-return or dart check valve to prevent accidental re-contamination of the wound with exudate.

The method described herein has the following significant advantages: it accelerates wound closure and/or reduces bacterial count in the wound beyond the rate of wound closure achievable with the VAC and povidone-iodine, while being completely compatible with wound tissue, and thus being not irritating to the wound as seen with povidone-iodine or silver salts.

More specifically, there is provided a treatment regimen for promoting wound healing of a mammalian patient having a wound, wherein the regimen comprises alternating a short-term application with a long-term application to the wound, wherein the short term application comprises contacting the wound with a cell-compatible aganocide composition described herein in an amount effective to benefit wound healing, and wherein the long-term application comprises contacting the wound with an effective amount of the composition to promote wound healing, and wherein the long-term application is being carried out in time intervals to promote wound healing and appropriate to the patient in need of wound care, while sufficient negative pressure is applied to the wound for a sufficient time period in conjunction with, or before, during or after application of the aganocide composition, to accelerate the wound healing process.

In general, the regimen may be carried out until (i) wound closure is occurring or (ii) the size of the wound and/or the bacterial load have been reduced sufficiently to allow self-healing of the wound. While there is no time limit for the regimen, in general a regimen over about 2-4 weeks will be sufficient to achieve sufficient wound closure or a satisfactory reduction of the bacterial load of the wound to promote wound healing. The regimen is preferred wherein the short term application and the long-term application are separated by removal of the aganocide composition from the wound. However, over the entire cycle of alternations or the wound-treating cycle it is immaterial whether the cycle started with a short-term or a long-term application of the aganocide, although a treatment regimen starting with a short-term application of the aganocide will be preferred, because it is likely to produce more quickly beneficial results. Good results are achievable if the first negative pressure cycle commences after an initial treatment of the wound with the aganocide composition. In one preferred procedure, after each negative pressure cycle, organic aganocide or an organic aganocide source or a saline composition can be placed again on the wound and any remaining wound debris be removed by gentle atraumatic wiping. The vacuum system may also be connected via a valve with a container holding aganocide as described herein. This would allow aganocide treatment between successive negative pressure cycles. Alternatively, the treatment regimen, although this is less preferred, may begin-with a negative pressure step, followed by application of the aganocide, and repeated alternations of this treatment sequence.

The combination treatment described herein decreases fluid excess and edema, and thereby bacterial colonization. In sternal wound infections after cardiac surgery the ICU (Intensive Care Unit) stay of a patient may be significantly shortened by utilizing this combination treatment regimen. In addition, the risk of sepsis due to bacteria swept into the blood circulation is markedly decreased.

The organic aganocide may also be applied in conjunction with an inorganic aganocide such as HOCl, an HOCl source, or mixtures thereof. If an organic and an inorganic aganocide are applied in the method of the invention, preferably, the effective amount of hypochlorous acid is from about 20 to about 500 ppm. Even more preferred, the effective amount is from about 40 to about 250 ppm. Most preferably, the effective amount is from about 50 to about 150 ppm.

### EMBODIMENTS AND ASPECTS OF THE INVENTION

In one embodiment of the application, there is provided a method for the treatment of a chronic wound using a wound treatment regimen, the regimen comprising: a) applying reduced pressure to the wound; b) treating the wound with a substantially non-toxic, non-irritating organic aganocide composition while applying reduce pressure to the wound; c) optionally, treating the wound with saline after at least one aganocide treatment step, wherein steps a), b) and c) together comprising a regimen cycle; and d) optionally, repeating at least one additional treatment regimen cycle. In one variation of the method, the organic aganocide composition is selected from the group consisting of an N-halo amino acid, an N,N-dihaloamino acid, a source of an N-halo amino acid, a source of an N,N-dihaloamino acid and mixtures thereof. In another variation, the treatment of the aganocide to the wound is performed before the application of reduced pressure to the wound. In another variation of the method, the aganocide treatment of the wound is done after a first application of reduced pressure to the wound. In yet another variation, the saline treatment is performed in conjunction with applying the reduced pressure. In a particular variation of the above method, the aganocide treatment and the application of reduced pressure is done intermittently. In another variation of the method, each aganocide treatment step is followed by a reduced pressure application step, followed by a second aganocide treatment step, followed by a second reduced pressure application step, this sequence of steps being considered a single treatment cycle. In yet another variation, the method comprises a single treatment cycle or at least two single treatment cycles. In one variation of the above method, the method comprises at least four, six, ten, twelve or 24 but not more than 48 single treatment cycles. In another variation of each of the above method, the aganocide is an N-halo amino acid or an N,N-dihaloamino acid. In yet another variation of the method, the treatment with the aganocide is performed by an automated delivery system. In a particular variation, the N-halo amino acid or an N,N-dihaloamino acid composition has a pH from about 3.5 to 7.

In another embodiment of the present application, there is provided a method for the treatment of a chronic wound using a wound treatment regimen, the regimen comprising: a) treating the wound with at least one application of a substantially non-toxic, non-irritating organic aganocide composition; b) treating the wound with saline; and c) applying reduced pressure to the wound. In one variation of the method, the organic aganocide composition is selected from the group consisting of an N-halo amino acid, an N,N-dihaloamino acid, a source of an N-halo amino acid, a source of an N,N-dihaloamino acid and mixtures thereof. In another variation, the treatment of the aganocide to the wound is performed before the application of reduced pressure to the wound. In yet another variation, the aganocide treatment of the wound is done after a first application of reduced pressure to the wound. In one particular variation, the saline treatment is performed in conjunction with applying the reduced pressure. In another particular variation, the aganocide treatment and the application of reduced pressure is done intermittently. In another variation of the above method, each aganocide treatment step is followed by a reduced pressure application step, followed by a second aganocide treatment step, followed by a second reduced pressure application step, this sequence of steps being considered a single treatment cycle. In another variation of the above method, the method provides a single treatment cycle or at least two single treatment cycles. In another variation, the method comprises at least four, six, ten, twelve or 24 but not more than 48 single treatment cycles. In a particular variation, the treatment of the wound with saline is applied after at least one aganocide treatment and one reduced pressure application step.

In another embodiment of the present application, there is provided a method for promoting wound healing of a mammalian patient having a wound using a treatment regimen, wherein the regimen comprises: a) a short term application comprising contacting the wound with a cell-compatible aganocide composition in an amount effective to promote wound healing; b) a long term application comprising contacting the wound with an effective amount of the aganocide composition to promote wound healing, wherein the short term application and the long term application are performed at alternating time intervals to promote wound healing and appropriate to the patient in need of wound care; and c) applying reduced pressure to the wound for a sufficient period of time to promote wound healing. In one variation of the method, the organic aganocide composition is selected from the group consisting an N-chlorotaurine, N,N-dichlorotaurine and a compound of the formula
A-C(R¹R⁰)R(CH₂)ₙ—C(YZ)—X′ (I)
or a derivative thereof, wherein A is hydrogen, HalNH— or Hal₂N— wherein Hal is halogen selected from the group consisting of chloro and bromo; R is a carbon carbon single bond or a divalent cycloalkylene radical with three to six carbon atoms, R¹ is hydrogen, lower alkyl or the group —COOH; R⁰ is hydrogen or lower alkyl; n is 0 or an integer from 1 to 13, or R¹ and R⁰ together with the carbon atom to which they attach form a (C₃-C₆)cycloalkyl ring; Y is hydrogen, lower alkyl or —NH₂ or —NHal₂; and Z is hydrogen or lower alkyl; and X′ is hydrogen, —COOH, —CONH₂, —SO₃H, —SO₂NH₂, or —P(═O)(OH)₂, a salt thereof, a derivative thereof, a N,N-dihaloamino acid of U.S. Pat. No. 4,386,103, and mixtures thereof.

In a particular variation of the above method, the short term and/or long term application of the aganocide to the wound is performed before the application of reduced pressure to the wound. In one variation of the above, the short term and/or long term application of the aganocide to the wound is done after a first application of reduced pressure to the wound. In another variation, the saline treatment is performed in conjunction with applying the reduced pressure. In yet another variation of the above method, the short term and/or the long term application of the aganocide and the application of reduced pressure is done intermittently. In another variation, each of the short term and/or each of the long term aganocide application step is followed by a reduced pressure application step, followed by a second short term and/or long term aganocide application step, followed by a second reduced pressure application step, this sequence of steps being considered a single treatment cycle. In yet another variation, of the method, the method comprises one treatment cycle or at least two single treatment cycles. In one variation, the method comprises at least four, six, ten, twelve or 24 but not more than 48 single treatment cycles. In another variation of each of the above methods, the aganocide is selected from the group consisting of N-chlorotaurine, N,N-dichlorotaurine, N,N-dichloro-2,2-dimethyltaurine, a salt thereof, and mixtures thereof. In another variation, the treatment with the aganocide is performed by an automated delivery system. In yet another variation, the aganocide composition has a pH from about 3.5 to 7. In another variation, the aganocide further includes HOCl or an HOCl source. In one variation of the above, the amount of aganocide is between about 100 to about 10,000 ppm, about 200 ppm to about 2,000 ppm, or about 400 ppm to about 1,000 ppm. In another variation, the amount of HOCl is from about 20 ppm to 500 ppm, about 40 ppm to 250 ppm or about 50 ppm to 150 ppm. In yet another variation, the pH of the aganocide composition is from about 2 to 9.5, or about 3.5 to 6.5.

### PREFERRED EMBODIMENTS

In a particular aspect, it is preferred to automate the delivery of a topical solution of the organic aganocide composition described herein and its removal from the wound because it assists with wound cleansing, irrigation and removal of infectious materials and minimizes costly and time-consuming caregiver intervention. As described above, if the aganocide composition of the invention is administered during the application of the VAC system, and if its administration and removal is automated, the VAC system can be used in a homecare setting and human caregiver's time involvement may be limited to teaching and training the patient to use such an automated VAC system. Details of this combination therapy are described in KCI's V.A.C.®^{instill} brochure, the entire text of which is incorporated herein by reference (hhtp://www.kcil.com/29-A-142InstillBrochure12-05.pdf).

If an N-halo- or N,N-dihalo amino acid is used as the aganocide, at the preferred pH range described herein, the aganocide is present in a salt form, preferably in an inorganic salt form, such as the sodium, potassium, magnesium, calcium, aluminum, etc. salt. The most preferred salt is sodium 2-dichloroamino-2-methylpropanesulfonate.

The V.A.C.®^{instill} system is perfectly suitable to deliver the aganocide compositions of the present invention in an automated fashion. A complete treatment cycle may work as follows, although other treatment cycles are also suitable:

| Mode | Time Range |
|---|---|
| Instillation of aganocide composition; | 1 second-10 minutes, |
| Example: 500 ppm N,N-dichloro-2,2-dimethyl | preferably |
| taurine in 0.9% saline solution, | 1 second-5 minutes |
| Leave composition on the wound | 1 second-2 hours, |
| | preferably |
| | 30 seconds-1 hour |
| Apply Vacuum Therapy | 1 minute-24 hours, |
| | preferably |
| | 5 minutes-12 hours |

However, the combination therapy as described herein is not limited to the sequence of modes or time ranges shown in the table above. For example, the treatment could begin with mechanical treatment of the wound with the aganocide, for example, by pouring it on before use of the V.A.C. system. Alternatively, the treatment could begin by applying negative pressure first, followed by aganocide treatment etc.

In comparison with Dakin's solution hypochlorous acid exhibits improved antimicrobial activity, and improved therapeutic index and improved time kill in the regimen described herein. This means that the time to wound closure may be reduced using hypochlorous acid.

### EXAMPLE 1

### Sodium 2-dichloroamino-2-methylpropanesulfonate

A human secondary intention wound approximately 3-4 cm in diameter is fitted with a polyurethane sponge, pore size 400-600 microns cut to the size of the wound and twice the thickness of the wound and covered with an adhesive film, per instructions on the vacuum system. The outlet is connected to an IV bag (500 ml) with a 0.1% sodium 2-dichloroamino-2-methylpropanesulfonate solution adjusted with HCl to a pH of about 4, 0.9% NaCl and elevated to between 0.1 and 1 meter above the wound. A 1-cm hole is punctured in the adhesive film, and a second adhesive film is used to cover the hole, with the second outlet connected to the vacuum pump.

Each cycle consists of three phases: a 2-hour pumping phase when the vacuum (125 mm Hg negative pressure) is connected and the IV bag is disconnected; a 1 second instilling phase when the vacuum is disconnected and the IV bag is connected and the aganoocide is applied to the wound; and a 1 minute holding phase when both vacuum and the IV bag are disconnected.

Each treatment has about 24 cycles and starts with a pumping phase and proceeds for 48 hours. When the treatment is completed, the dressings are changed and the treatment is repeated until full granulation has been promoted and the granulation tissue is within the level of the skin, approximately 30 to 100 days, depending on the depth of the original wound.

When compared with treatment of a similar wound with vacuum-assisted wound healing without aganocide or treatment of the wound with aganocide alone the combination treatment regimen described herein achieves superior wound healing results.

### EXAMPLE 2

### Sodium 2-dichloroamino-2-methylpropanesulfonate

A human secondary intention wound approximately 3-4 cm in diameter is fitted with a polyurethane sponge, pore size 400-600 microns cut to the size of the wound and twice the thickness of the wound and covered with an adhesive film, per instructions on the vacuum system. The outlet is connected to an IV bag (500 ml) with a 0.1% sodium 2-dichloroamino-2-methylpropanesulfonate solution adjusted with HCl to a pH of about 4, 0.9% NaCl, and is elevated to between 0.1 and 1 meter above the wound. A 1-cm hole is punctured in the adhesive film, and a second adhesive film is used to cover the hole, with the second outlet connected to the vacuum pump.

Each cycle consists of three phases: a 2-hour pumping phase when the vacuum (125 mm Hg negative pressure) is connected and the IV bag is disconnected; a 5 second instilling phase when the vacuum is disconnected and the IV bag is connected and the aganocide is applied to the wound; and a 30 minute holding phase when both vacuum and the IV bag are disconnected.

Each treatment has about 19 cycles and starts with a pumping phase and proceeds for 48 hours. When the treatment is completed, the dressings are changed and the treatment is repeated until full granulation has been promoted and the granulation tissue is within the level of the skin, approximately 30 to 100 days, depending on the depth of the original wound.

When compared with treatment of a similar wound with vacuum-assisted wound healing without aganocide or treatment of the wound with aganocide alone the combination treatment regimen described herein achieves superior wound healing results.

The entire disclosures of all patents, patent publications and cited are references are incorporated herein by reference in their entirety.

What is claimed:

1. A method for the treatment of a chronic wound using a wound treatment regimen, the regimen comprising:
a) applying reduced pressure to the wound;
b) treating the wound with a substantially non-toxic, non-irritating organic aganocide composition while applying reduce pressure to the wound;
c) optionally, treating the wound with saline after at least one aganocide treatment step, wherein steps a), b) and c) together comprising a regimen cycle; and
d) optionally, repeating at least one additional treatment regimen cycle.

2. The method of claim 1, wherein the organic aganocide composition is selected from the group consisting of an N-halo amino acid, an N,N-dihaloamino acid, a source of an N-halo amino acid, a source of an N,N-dihaloamino acid and mixtures thereof.

3. The method of claim 1, wherein the treatment of the aganocide to the wound is performed before the application of reduced pressure to the wound.

4. The method of claim 1, wherein the aganocide treatment of the wound is done after a first application of reduced pressure to the wound.

5. The method of claim 1, wherein the saline treatment is performed in conjunction with applying the reduced pressure.

6. The method of claim 2, wherein aganocide treatment and the application of reduced pressure is done intermittently.

7. The method of claim 1, wherein each aganocide treatment step is followed by a reduced pressure application step, followed by a second aganocide treatment step, followed by a second reduced pressure application step, this sequence of steps being considered a single treatment cycle.

8. The method of claim 7, comprising a single treatment cycle or at least two single treatment cycles.

9. The method of claim 7, comprising at least four, six, ten, twelve or 24 but not more than 48 single treatment cycles.

10. The method of claim 1, wherein the aganocide is an N-halo amino acid or an N,N-dihaloamino acid.

11. The method of claim 1, wherein the treatment with the aganocide is performed by an automated delivery system.

12. The method of claim 10, wherein the N-halo amino acid or an N,N-dihaloamino acid composition has a pH from about 3.5 to 7.

13. A method for the treatment of a chronic wound using a wound treatment regimen, the regimen comprising:
a) treating the wound with at least one application of a substantially non-toxic, non-irritating organic aganocide composition;
b) treating the wound with saline; and
c) applying reduced pressure to the wound.

14. The method of claim 13, wherein the organic aganocide composition is selected from the group consisting of an N-halo amino acid, an N,N-dihaloamino acid, a source of an N-halo amino acid, a source of an N,N-dihaloamino acid and mixtures thereof.

15. The method of claim 13, wherein the treatment of the aganocide to the wound is performed before the application of reduced pressure to the wound.

16. The method of claim 13, wherein the aganocide treatment of the wound is done after a first application of reduced pressure to the wound.

17. The method of claim 13, wherein the saline treatment is performed in conjunction with applying the reduced pressure.

18. The method of claim 14, wherein aganocide treatment and the application of reduced pressure is done intermittently.

19. The method of claim 13, wherein each aganocide treatment step is followed by a reduced pressure application step, followed by a second aganocide treatment step, followed by a second reduced pressure application step, this sequence of steps being considered a single treatment cycle.

20. The method of claim 19, comprising a single treatment cycle or at least two single treatment cycles.

21. The method of claim 19, comprising at least four, six, ten, twelve or 24 but not more than 48 single treatment cycles.

22. The method of claim 13, wherein the treatment of the wound with saline is applied after at least one aganocide treatment and one reduced pressure application step.

23. A method for promoting wound healing of a mammalian patient having a wound using a treatment regimen, wherein the regimen comprises:
a) a short term application comprising contacting the wound with a cell-compatible aganocide composition in an amount effective to promote wound healing;
b) a long term application comprising contacting the wound with an effective amount of the aganocide composition to promote wound healing, wherein the short term application and the long term application are performed at alternating time intervals to promote wound healing and appropriate to the patient in need of wound care; and
c) applying reduced pressure to the wound for a sufficient period of time to promote wound healing.

24. The method of claim 23, wherein the organic aganocide composition is selected from the group consisting an N-chlorotaurine, N,N-dichlorotaurine and a compound of the formula
A-C(R¹R⁰)R(CH₂)ₙ—C(YZ)—X′ (I)
or a derivative thereof, wherein A is hydrogen, HalNH— or Hal₂N— wherein Hal is halogen selected from the group consisting of chloro and bromo; R is a carbon single bond or a divalent cycloalkylene radical with three to six carbon atoms, R¹ is hydrogen, lower alkyl or the group —COOH; R⁰ is hydrogen or lower alkyl; n is 0 or an integer from 1 to 13, or R¹ and R⁰ together with the carbon atom to which they attach form a (C₃-C₆)cycloalkyl ring; Y is hydrogen, lower alkyl or —NH₂ or —NHal₂; and Z is hydrogen or lower alkyl; and X′ is hydrogen, —COOH, —CONH₂, —SO₃H, —SO₂NH₂ or —P(═O)(OH)₂, a salt thereof, a derivative thereof, and mixtures thereof.

25. The method of claim 23, wherein the short term and/or long term application of the aganocide to the wound is performed before the application of reduced pressure to the wound.

26. The method of claim 23, wherein the short term and/or long term application of the aganocide to the wound is done after a first application of reduced pressure to the wound.

27. The method of claim 23, wherein the saline treatment is performed in conjunction with applying the reduced pressure.

28. The method of claim 24, wherein short term and/or the long term application of the aganocide and the application of reduced pressure is done intermittently.

29. The method of claim 23, wherein each of the short term and/or each of the long term aganocide application step is followed by a reduced pressure application step, followed by a second short term and/or long term aganocide application step, followed by a second reduced pressure application step, this sequence of steps being considered a single treatment cycle.

30. The method of claim 29, comprising one treatment cycle or at least two single treatment cycles.

31. The method of claim 29, comprising at least four, six, ten, twelve or 24 but not more than 48 single treatment cycles.

32. The method of claim 23, wherein the aganocide is selected from the group consisting of N-chlorotaurine, N,N-dichlorotaurine, N, N-dichloro-2,2-dimethyltaurine, a salt thereof, and mixtures thereof.

33. The method of claim 23, wherein the treatment with the aganocide is performed by an automated delivery system.

34. The method of claim 32, wherein the aganocide composition has a pH from about 3.5 to 7.

35. The method of claim 32, wherein the aganocide further includes HOCl or an HOCl source.

36. The method of claim 32, wherein the amount of aganocide is between about 100 to about 10,000 ppm, about 200 ppm to about 2,000 ppm, or about 400 ppm to about 1,000 ppm.

37. The method of claim 35, wherein the amount of HOCl is from about 20 ppm to 500 ppm, about 40 ppm to 250 ppm or about 50 ppm to 150 ppm.

38. The method of claim 29, wherein the pH of the aganocide composition is from about 2 to 9.5, or about 3.5 to 6.5.
